(19) United States
(12) Patent Application Publication
(10) Pub. No.: US 2021/0181457 A1
(43) Pub. Date: Jun. 17, 2021

(54) OPTICAL DEVICE AND ENDOSCOPE

(71) Applicant: OLYMPUS CORPORATION, Tokyo (JP)

(72) Inventor: Takahiro SHIMONO, Tokyo (JP)

(73) Assignee: OLYMPUS CORPORATION, Tokyo (JP)

(21) Appl. No.: 17/183,475

(22) Filed: Feb. 24, 2021

Related U.S. Application Data
(63) Continuation of application No. PCT/JP2019/005364, filed on Feb. 14, 2019.

(30) Foreign Application Priority Data
Aug. 27, 2018 (JP) 2018-158411

(51) Int. Cl. G02B 7/10; A61B 1/00; G02B 23/24

(52) U.S. Cl. CPC G02B 7/10; A61B 2017/00876; G02B 23/2476; A61B 1/00188

(57) ABSTRACT

An optical device includes a moving lens frame formed of a magnetic body, a fixed lens frame holding the moving lens frame on an inner peripheral surface so that the moving lens frame is movable along an optical axis, a first magnet and a second magnet disposed separately from each other in a direction of the optical axis on an outer periphery of the fixed lens frame and each having an annular shape, and a coil wound between the first magnet and the second magnet on the outer periphery of the fixed lens frame, and the moving lens frame has a non-rotationally symmetric shape about the optical axis.

### CROSS REFERENCE TO RELATED APPLICATION

This application is a continuation application of PCT/JP2019/005364 filed on Feb. 14, 2019 and claims benefit of Japanese Application No. 2018-158411 filed in Japan on Aug. 27, 2018, the entire contents of which are incorporated herein by this reference.

### BACKGROUND OF THE INVENTION

### 1. Field of the Invention

The present invention relates to an optical device capable of changing an optical function by moving a moving frame holding an optical member in an optical axis direction by magnetic force.

### 2. Description of the Related Art

In a conventionally known image pickup apparatus for which downsizing is requested, in particular, such as a portable terminal equipped with a camera or an endoscope, a two-focal-point switching optical device capable of switching the focal point of an image pickup optical system by using a magnetic actuator has been employed.

In such an optical device, a predetermined clearance is provided between an inner peripheral surface of a fixed frame and an outer peripheral surface of a moving frame so that the moving frame can slide inside the fixed frame. However, such a clearance potentially causes backlash of the moving frame and affects an optical property.

To avoid this issue, for example, Japanese Patent Application Laid-Open Publication No. 2017-63845 discloses a technology of an optical device including: an objective lens; a moving lens; a moving frame made of a magnetic body; a holding frame holding the moving frame so that the moving frame can freely move forward and backward; a pair of magnets provided on an outer periphery of the holding frame; a yoke provided between the pair of magnets; and a coil provided closer to the holding frame than the yoke. The yoke includes a frame part that covers an outer periphery of the coil, and a yoke convex portion partially formed in a circumferential direction of the frame part and protruding at an outer peripheral surface of the holding frame at front and back ends of the frame part to reduce a distance between the frame part and the outer peripheral surface. Accordingly, backlash when the moving frame is moved by using magnetic force is prevented with a simple configuration.

### SUMMARY OF THE INVENTION

An optical device according to an aspect of the present invention includes: an optical system including a moving lens that is movable in a direction of an optical axis; a moving frame formed of a magnetic body material and holding the moving lens; a holding frame formed of a non-magnetic body material in a tubular shape and holding the moving frame on an inner peripheral surface so that the moving frame is movable along the optical axis; a first magnet and a second magnet disposed separately from each other in the direction of the optical axis on an outer periphery of the holding frame and each having an annular shape; and a coil wound between the first magnet and the second magnet on the outer periphery of the holding frame. The moving frame has a non-rotationally symmetric shape about the optical axis.

An endoscope according to another aspect of the present invention includes an optical device including: an optical system including a moving lens that is movable in a direction of an optical axis; a moving frame formed of a magnetic body material and holding the moving lens; a holding frame formed of a non-magnetic body material in a tubular shape and holding the moving frame on an inner peripheral surface so that the moving frame is movable along the optical axis; a first magnet and a second magnet disposed separately from each other in the direction of the optical axis on an outer periphery of the holding frame and each having an annular shape; and a coil wound between the first magnet and the second magnet on the outer periphery of the holding frame. The moving frame has a non-rotationally symmetric shape about the optical axis.

### BRIEF DESCRIPTION OF THE DRAWINGS

FIG. 1 is a schematic configuration diagram of an endoscope;

FIG. 2 is a cross-sectional view illustrating a configuration of an image pickup apparatus in which a moving lens unit is moved to a distal end side;

FIG. 3 is a cross-sectional view illustrating the configuration of the image pickup apparatus in which the moving lens unit is moved to a proximal end side;

FIG. 4 is a perspective view of the moving lens unit;

FIG. 5 is an explanatory diagram schematically illustrating magnetic force received by a moving lens frame inside a fixed lens frame;

FIG. 6 is a perspective view of the moving lens unit according to a first modification;

FIG. 7 is a perspective view of the moving lens unit according to a second modification;

FIG. 8 is a main part cross-sectional view of an optical device according to a third modification at a section in a direction orthogonal to an optical axis;

FIG. 9 is a main part cross-sectional view of the optical device according to a fourth modification at a section in the direction orthogonal to the optical axis;

FIG. 10 is a main part cross-sectional view of the optical device according to a fifth modification at a section in the direction orthogonal to the optical axis;

FIG. 11 is a main part cross-sectional view of the optical device according to a sixth modification at a section in the direction orthogonal to the optical axis;

FIG. 12 is a main part cross-sectional view of the optical device according to a seventh modification at a section in the direction orthogonal to the optical axis;

FIG. 13 is a perspective view of the moving lens unit according to an eighth modification;

FIG. 14 is a perspective view of the moving lens unit according to a ninth modification; and

FIG. 15 is a perspective view of the moving lens unit according to a tenth modification.

### DETAILED DESCRIPTION OF THE PREFERRED EMBODIMENT

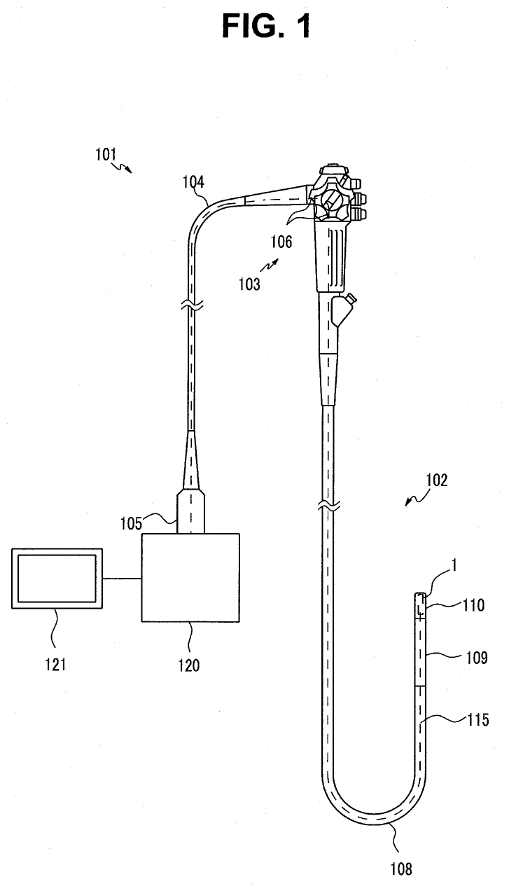

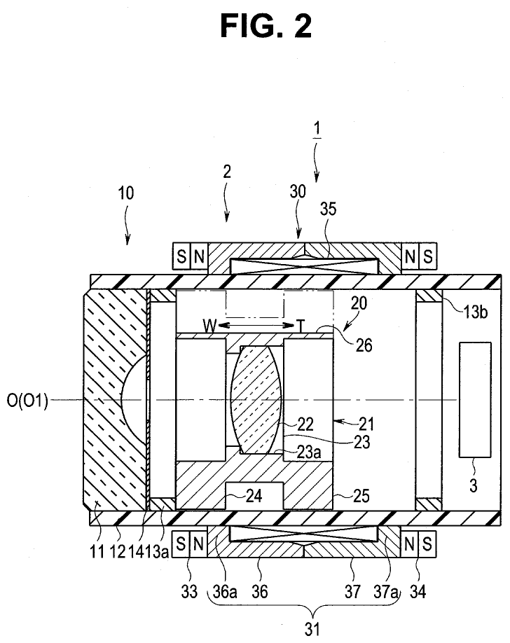

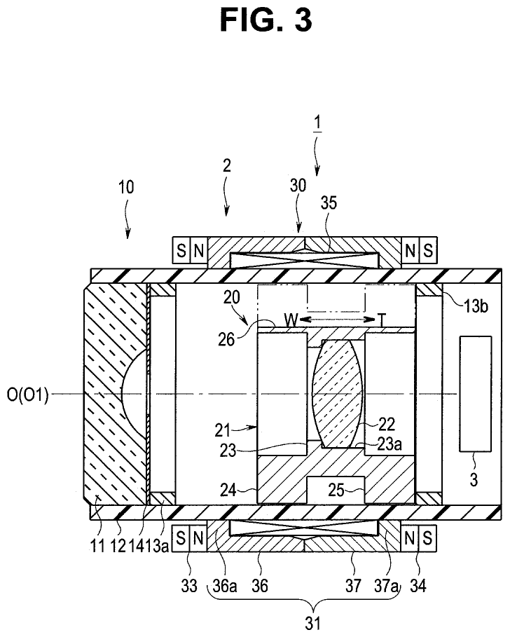

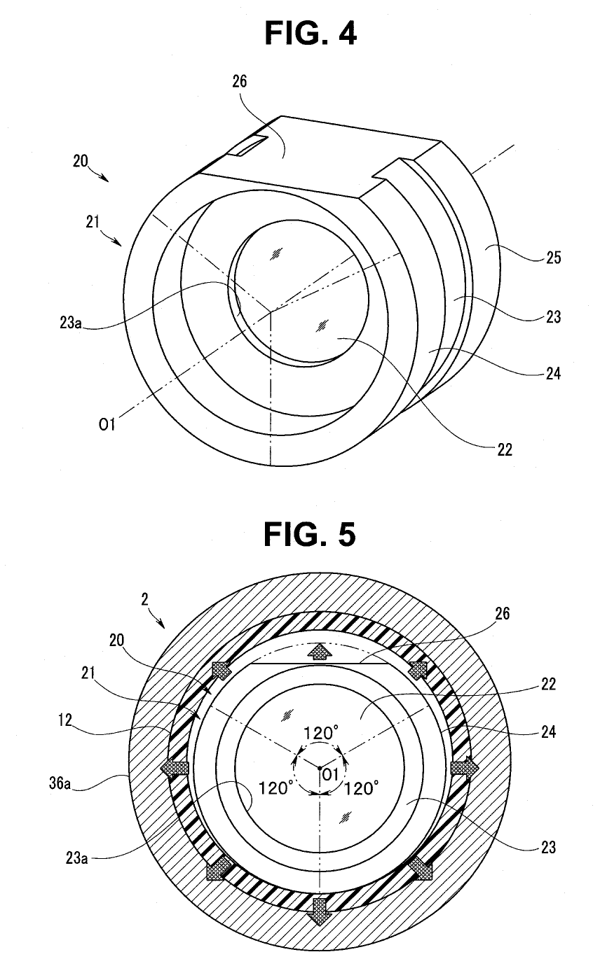

Forms of the present invention will be described below with reference to the accompanying drawings. The drawings relate to an embodiment of the present invention, FIG. 1 is a schematic configuration diagram of an endoscope, FIG. 2 is a cross-sectional view illustrating a configuration of an image pickup apparatus in which a moving lens unit is moved to a distal end side, FIG. 3 is a cross-sectional view illustrating the configuration of the image pickup apparatus in which the moving lens unit is moved to a proximal end side, FIG. 4 is a perspective view of the moving lens unit, and FIG. 5 is an explanatory diagram schematically illustrating magnetic force received by a moving lens frame inside a fixed lens frame.

An endoscope 101 of the present embodiment can be introduced into a subject such as a human body and is configured to optically pick up an image of a predetermined observation site in the subject.

Note that the subject into which the endoscope 101 is introduced is not limited to a human body but may be another living body or may be an artificial object such as a machine or a building.

The endoscope 101 mainly includes an insertion portion 102 that is introduced into the subject, an operation portion 103 positioned at a proximal end of the insertion portion 102, and a universal cord 104 extending from a side part of the operation portion 103.

The insertion portion 102 has a configuration in which a distal end portion 110 disposed at a distal end, a bending portion 109 that is bendable and disposed on the proximal end side of the distal end portion 110, and a flexible tube portion 108 that is flexible, disposed on the proximal end side of the bending portion 109, and connected with a distal end side of the operation portion 103 are continuously provided.

Although described later in detail, an image pickup apparatus 1 is provided at the distal end portion 110. An angle operation knob 106 for operating bending of the bending portion 109 is provided at the operation portion 103.

An endoscope connector 105 that is connected with an external device 120 is provided at a proximal end portion of the universal cord 104. The external device 120 that is connected with the endoscope connector 105 is connected with an image display unit 121 such as a monitor through a cable.

The endoscope 101 also includes a composite cable 115 inserted into the universal cord 104, the operation portion 103, and the insertion portion 102, and an optical fiber bundle (not illustrated) that transmits illumination light from a light source unit provided at the external device 120.

The composite cable 115 electrically connects the endoscope connector 105 and the image pickup apparatus 1. When the endoscope connector 105 is connected with the external device 120, the image pickup apparatus 1 is electrically connected with the external device 120 through the composite cable 115.

Electrical power supply from the external device 120 to the image pickup apparatus 1 and communication between the external device 120 and the image pickup apparatus 1 are performed through the composite cable 115.

An image processing unit is provided at the external device 120. The image processing unit generates a video signal based on an image pickup device output signal that is output from the image pickup apparatus 1, and outputs the generated video signal to the image display unit 121. Accordingly, in the present embodiment, an optical image (endoscope image) picked up by the image pickup apparatus 1 is displayed as a video on the image display unit 121.

Note that the endoscope 101 does not necessarily need to be connected with the external device 120 or the image display unit 121, but for example, may include part or whole of the image processing unit or the monitor.

The optical fiber bundle (not illustrated) has a configuration through which light emitted from the light source unit of the external device 120 is transmitted to an illumination window as an illumination light emission portion of the distal end portion 110. The light source unit may be disposed in the operation portion 103 or the distal end portion 110 of the endoscope 101.

Subsequently, the configuration of the image pickup apparatus 1 according to the present embodiment will be described below in detail.

As illustrated in FIGS. 2 and 3, the image pickup apparatus 1 includes, for example, a two-focal-point switching optical device 2, and an image pickup device 3 continuously provided on a back side of the optical device 2.

Note that the image pickup device 3 is an image sensor such as a CCD or a CMOS, fixed to an image pickup device holding frame (not illustrated), and continuously provided on the back side of the optical device 2 through the image pickup device holding frame.

The optical device 2 includes a fixed lens unit 10, a moving lens unit 20, and an actuator 30 as a drive mechanism.

The fixed lens unit 10 includes a fixed lens 11 that is an objective lens as an optical system through which light of an object image (optical image) is focused toward the image pickup device 3, a substantially tubular fixed lens frame 12 that is a lens holding frame as a holding frame formed of a non-magnetic member, and two restriction members 13a and 13b each having an annular shape. Note that one of the restriction members 13a and 13b may be part of the lens holding frame 12 (in other words, may be integrally formed with the lens holding frame 12).

The fixed lens frame 12 is formed in a tube elongated along an image pickup optical axis O and holds, at a distal end part of the image pickup optical axis O, the fixed lens 11 provided with an optical aperture 14 as an optical member included in the optical system. Note that the fixed lens 11 may be configured as a group of a plurality of lenses.

The restriction member 13a configured to restrict a position of the distal end side of the moving lens unit 20 to sandwich the optical aperture 14 on the back side of the fixed lens 11 is fixed to an inner peripheral part of the fixed lens frame 12. The restriction member 13b configured to restrict a position of a back end side of the moving lens unit 20 is fixed on a front side of the image pickup device 3.

The moving lens unit 20 includes a moving lens frame 21 as a moving frame formed of a magnetic member and having a substantially tubular shape as a basic shape, and a moving lens 22 as an optical member that is included in the optical system and through which light of the object image is focused to a light receiving unit of the image pickup device 3.

As illustrated in FIGS. 2 to 4, in the moving lens frame 21, a lens holding portion 23, a first sliding portion 24 as a sliding portion continuously provided on the distal end side of the lens holding portion 23, and a second sliding portion 25 as a sliding portion continuously provided on the proximal end side of the lens holding portion 23 are integrally formed of a magnetic body.

The lens holding portion 23 has a substantially annular shape having an inner periphery formed as a lens holding hole 23a, and the moving lens 22 is held in the lens holding hole 23a of the lens holding portion 23. Note that the moving lens 22 may be configured as a group of a plurality of lenses.

The first and second sliding portions 24 and 25 are provided continuously and coaxially on a central axis O1 of the lens holding hole 23a of the lens holding portion 23.

The first and second sliding portions 24 and 25 are each configured as a substantially annular member having an outer diameter larger than an outer diameter of the lens holding portion 23 and slightly smaller than an inner diameter of the fixed lens frame 12. Accordingly, outer peripheral surfaces of the first and second sliding portions 24 and 25 are set as sliding surfaces that are freely slidable relative to an inner peripheral surface of the fixed lens frame 12.

Note that in the present embodiment, the outer diameters of the sliding portions 24 and 25 are, for example, 1.3 mm to 1.5 mm approximately, which are smaller than the inner diameter of the fixed lens frame 12 by, for example, 0.02 mm approximately.

The moving lens unit 20 is enclosed in the fixed lens frame 12 of the fixed lens unit 10 and is provided to be freely movable in a front-back direction along the image pickup optical axis O as the sliding portions 24 and 25 slide.

The moving lens frame 21 of the present embodiment includes a cutout portion 26 formed by cutting out part of the lens holding portion 23 and the first and second sliding portions 24 and 25 into an integrated planar shape by machining or the like in a direction orthogonal to the central axis O1, and accordingly, the moving lens frame 21 has a non-rotationally symmetric shape about the central axis O1.

In this case, for example, as illustrated in FIG. 5, the cutout portion 26 is formed on circumferences of the first and second sliding portions 24 and 25 in a range having a central angle of 120° or smaller.

With this configuration, the sliding surface of each of the sliding portions 24 and 25 can contact the inner peripheral surface of the fixed lens frame 12 at at least three points equally spaced from each other by 120° about the central axis O1, which enables stable forward-and-backward movement of the moving lens unit 20 in the fixed lens frame 12.

The actuator 30 includes a yoke 31 provided on an outer periphery of the fixed lens frame 12, a first magnet 33 having an annular shape and integrally fixed to a front end part of the yoke 31, a second magnet 34 having an annular shape and integrally fixed to a back end part of the yoke 31, and a solenoid coil (hereinafter simply referred to as coil) 35 wound and fixed between an inner periphery of the yoke 31 and the outer periphery of the fixed lens frame 12.

The yoke 31 of the present embodiment is formed in divisions of a first yoke member 36 and a second yoke member 37 each having a substantially cylindrical shape. An inward flange 36a is formed on an entire circumference at a distal end of the first yoke member 36, and the first magnet 33 is fixed to the inward flange 36a. In addition, an inward flange 37a is formed on an entire circumference at a back end of the second yoke member 37, and the second magnet 34 is fixed to the inward flange 37a. The first yoke member 36 and the second yoke member 37 are externally inserted from the distal end side and the back end side, respectively, of the fixed lens frame 12 to entirely enclose and cover the coil 35 and are connected and fixed so that facing end parts of the first yoke member 36 and the second yoke member 37 are integrated with each other by a bonding agent, brazing, or the like.

The first yoke member 36 and the second yoke member 37 are magnetic members such as soft iron for amplifying magnetic force generated at the first magnet 33, the second magnet 34, and the coil 35.

Note that the first yoke member 36 and the second yoke member 37 are formed as separate bodies for assembly to cover the coil 35 wound and fixed on the outer periphery of the fixed lens frame 12, but may be formed as one integrated yoke as long as assembly to cover the coil 35 is possible.

In the actuator 30, the first magnet 33 is a permanent magnet having a south pole magnetized on the front side and a north pole magnetized on the back side, and the second magnet 34 is a permanent magnet having a north pole magnetized on the front side and a south pole magnetized on the back side. In other words, the first magnet 33 and the second magnet 34 are disposed so that the same magnetic poles (north poles, in this example) face each other.

The actuator 30 thus configured can generate drive power to the moving lens unit 20 by switching a direction of energization to the coil 35 to switch a direction of the magnetic field generated at the coil 35 relative to the magnetic field of the first magnet 33 and the magnetic field of the second magnet 34.

Specifically, when the coil 35 is energized in a first direction in which the coil 35 is excited to have a south pole on the distal end side and a north pole on the proximal end side, the magnetic field of the first magnet 33 is strengthened on the distal end side of the actuator 30 by the magnetic field generated at the coil 35 in the same direction. Simultaneously, the magnetic field of the second magnet 34 is canceled and weakened on the back end side of the actuator 30 by the magnetic field generated at the coil 35 in the opposite direction.

As a result, drive power (magnetic force) that attracts the moving lens frame 21 toward the distal end side is generated at the actuator 30 as a whole, and moves the moving lens frame 21 (moving lens unit 20) to a front-side movement position defined by the restriction member 13a.

Note that after the energization to the coil 35 is canceled, the moving lens frame 21 after being moved to the distal end side is held by magnetic force of the first magnet 33.

When the coil 35 is energized in a second direction in which the coil 35 is excited to have a north pole on the distal end side and a south pole on the proximal end side, the magnetic field of the first magnet 33 is canceled and weakened on the distal end side of the actuator 30 by the magnetic field generated at the coil 35 in the opposite direction. Simultaneously, the magnetic field of the second magnet 34 is strengthened on the back end side of the actuator 30 by the magnetic field generated at the coil 35 in the same direction.

As a result, drive power (magnetic force) that attracts the moving lens frame 21 to the proximal end side is generated at the actuator 30 as a whole, and moves the moving lens frame 21 (moving lens unit 20) to a back-side movement position defined by the restriction member 13b.

Note that after the energization to the coil 35 is canceled, the moving lens frame 21 after being moved to the back end side is held by magnetic force of the second magnet 34.

Note that, in the image pickup apparatus 1, a state in which the moving lens unit 20 is moved forward and stopped on the front side is a wide end as a first stop position, and a state in which the moving lens unit 20 is moved backward and stopped on the back side is a tele end as a second stop position.

In this manner, the image pickup apparatus 1 has a configuration in which the moving lens unit 20 is moved forward and backward by drive of the actuator 30 to perform switching between two wide and tele optical properties.

The two wide and tele optical properties of the image pickup apparatus 1, which depend on the front and back stop positions of the moving lens unit 20, may be inverted by lens designing of the fixed lens 11 and the moving lens 22 and the like.

In movement and holding of the moving lens frame 21 (moving lens unit 20) as described above to and at the front-side movement position or in movement and holding of the moving lens frame 21 (moving lens unit 20) to and at the back-side movement position, magnetic force received by the moving lens frame 21 from the first and second magnets 33 and 34 and the coil 35 in each direction orthogonal to the central axis is ununiform since the moving lens frame 21 has a non-rotationally symmetric shape about a central axis of the lens holding portion 23 (lens holding hole 23a).

Specifically, a volume of a site at which the cutout portion 26 is provided is smaller than a volume of another site, and thus magnetic force (attraction force) received by the moving lens frame 21 at the site at which the cutout portion 26 is provided is relatively smaller than magnetic force (attraction force) received by the moving lens frame 21 at a site in another direction (refer to FIG. 5).

Accordingly, in the moving lens frame 21 (moving lens unit 20), a site opposite to the site at which the cutout portion 26 is provided is attracted and pressed toward the inner peripheral surface of the fixed lens frame 12. When the moving lens frame 21 is attracted in this manner to perform what is called backlash reduction, backlash of the moving lens unit 20 is appropriately prevented.

In this case, the cutout portion 26 of the moving lens frame 21 is desirably set with taken into account gravitational force received by the moving lens unit 20. Specifically, a cutout amount of the cutout portion 26 is desirably set so that a difference (relative attraction force) between attraction force that the moving lens frame 21 is attracted in a direction opposite to the cutout portion 26 by magnetic force and attraction force that the moving lens frame 21 is attracted in a direction toward the cutout portion 26 by magnetic force is larger than the gravitational force.

In addition, for example, as illustrated in FIGS. 2 and 3, the moving lens frame 21 is desirably designed so that the central axis O1 when backlash reduction of the moving lens frame 21 is performed is aligned with a central axis O of the optical device 2.

According to such an embodiment, the moving lens frame 21 formed of a magnetic body and including the lens holding hole 23a holding the moving lens 22, the fixed lens frame 12 formed of a non-magnetic body in a tubular shape and holding the moving lens frame 21 on an inner peripheral surface so that the moving lens frame 21 is movable along an optical axis O, the first magnet 33 and the second magnet 34 disposed at a predetermined distance from each other in a direction of the optical axis O on the outer periphery of the fixed lens frame 12 and each having an annular shape, and the coil 35 wound between the first magnet 33 and the second magnet 34 on the outer periphery of the fixed lens frame 12 are provided, and the moving lens frame 21 has a non-rotationally symmetric shape about the central axis O1 so that magnetic force received from the first and second magnets 33 and 34 and the coil 35 at a site in at least one direction orthogonal to the central axis O1 of the lens holding hole 23a is relatively smaller than magnetic force received from the first and second magnets 33 and 34 and the coil 35 at another site, thereby moving the moving lens frame 21 with sufficient drive power and appropriately preventing backlash of the moving lens frame 21.

Specifically, since the moving lens frame 21 has a non-rotationally symmetric shape about the central axis O1 and is configured so that magnetic force received from the first and second magnets 33 and 34 and the coil 35 at a site in at least one direction orthogonal to the central axis O1 is relatively smaller than magnetic force received from the first and second magnets 33 and 34 and the coil 35 at another site, backlash reduction of the moving lens frame 21 can be performed without increasing or decreasing the magnetic forces of the first and second magnets 33 and 34 only at biased parts during energization of the coil 35. In addition, sufficient drive power can be generated for the moving lens frame 21 by increasing or decreasing the magnetic forces of the first and second magnets 33 and 34 over the entire circumference during energization of the coil 35.

In this case, since the cutout portion 26 is formed by cutting out part of the lens holding portion 23 and the first and second sliding portions 24 and 25 into a planar shape, the moving lens frame 21 can be easily formed into a non-rotationally symmetric shape.

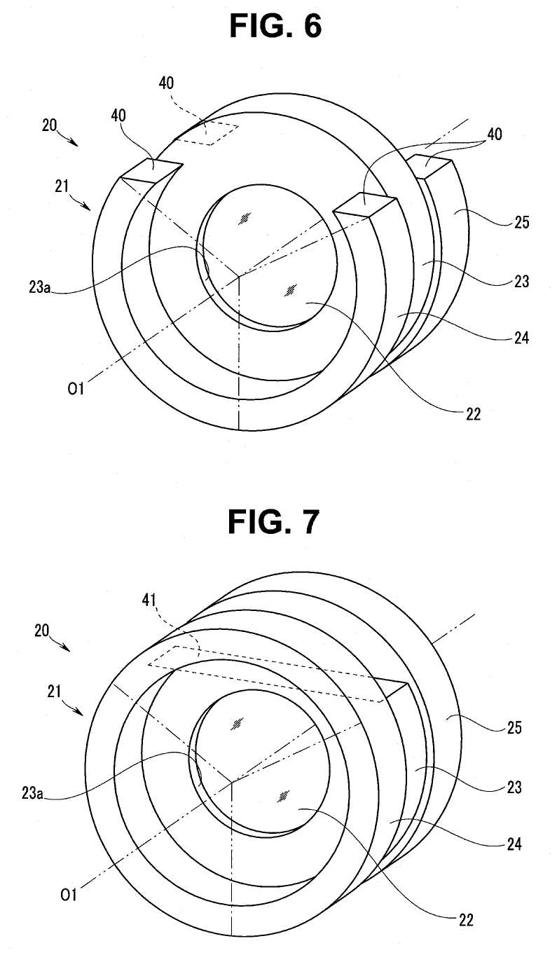

For example, as illustrated in FIG. 6, a cutout portion 40 may be formed only at the first and second sliding portions 24 and 25 among the lens holding portion 23 and the first and second sliding portions 24 and 25 in the moving lens frame 21.

With this configuration, the cutout portion can be more deeply formed while holding strength of the moving lens 22 is maintained, which improves the degree of freedom of attraction force amount designing for backlash reduction of the moving lens frame 21.

For example, as illustrated in FIG. 7, a cutout portion 41 may be formed only at the lens holding portion 23 among the lens holding portion 23 and the first and second sliding portions 24 and 25 in the moving lens frame 21.

With this configuration, it is possible to prevent backlash of the moving lens frame 21 while maintaining slidability of the first and second sliding portions 24 and 25 relative to an inner periphery of the fixed lens frame 12.

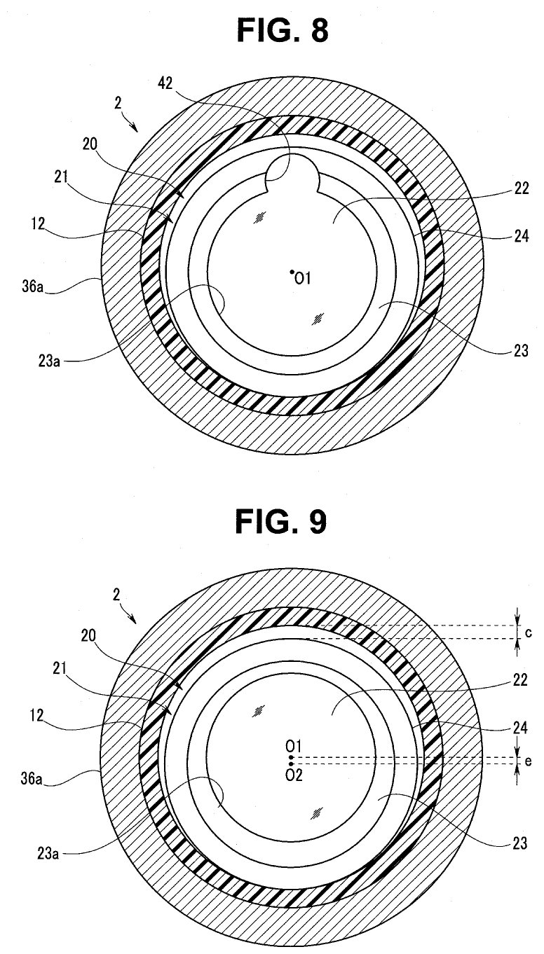

For example, as illustrated in FIG. 8, a hole portion 42 may be drilled through the moving lens frame 21 in place of the cutout portion, thereby forming a non-rotationally symmetric shape of the moving lens frame 21 about the central axis O1.

With this configuration, it is possible to prevent backlash of the moving lens frame 21 without affecting an outer shape of the moving lens frame 21.

For example, as illustrated in FIG. 9, the central axis O1 of the lens holding hole 23a may be decentered relative to a central axis O2 of the first and second sliding portions 24 and 25 and the like, thereby forming a non-rotationally symmetric shape of the moving lens frame 21 about the central axis O1.

In this case, a decentering amount e of the central axis O1 relative to the central axis O2 is set to be equal to a half value of a maximum clearance c (which is e=c/2) when the moving lens frame 21 is pressed against the inner periphery of the fixed lens frame 12, thereby achieving backlash reduction of the moving lens frame 21 so that the central axis O1 is constantly positioned at a center of the fixed lens frame 12 when the moving lens frame 21 rotates inside the fixed lens frame 12. Thus, the optical axis O and the central axis O1 can be constantly aligned with each other by setting the optical axis O of the optical device 2 at the center of the fixed lens frame 12.

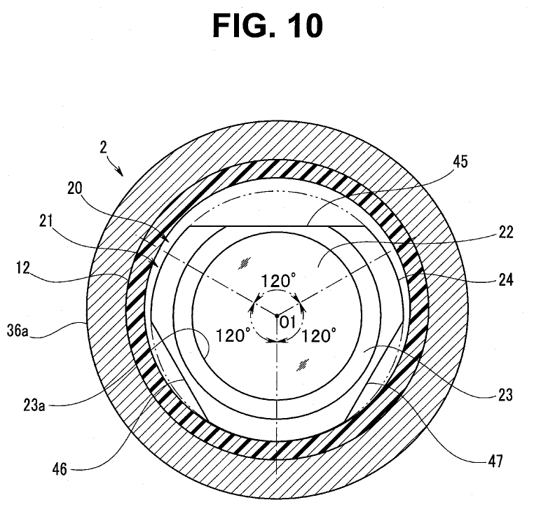

A plurality of cutout portions may be provided at the moving lens frame 21. For example, FIG. 10 illustrates a configuration of the moving lens frame 21 in which a first cutout portion 45 is provided at a site in one direction orthogonal to the central axis O1, and second and third cutout portions 46 and 47 smaller than the first cutout portion 45 are provided at rotational positions at 120° intervals with respect to the first cutout portion 45 about the central axis O1.

With this configuration, the magnetic forces received from the first and second magnets 33 and 34 and the coil 35 are smallest at the site at which the first cutout portion 45 is provided on a periphery of the moving lens frame 21, and is next smallest at sites at which the second and third cutout portions 46 and 47 are provided. Thus, the moving lens frame 21 is subjected to backlash reduction in a direction opposite to the first cutout portion 45 through the central axis O1 and is attracted toward sides opposite to the second and third cutout portions 46 and 47 through the central axis O1. Accordingly, the moving lens frame 21 is appropriately prevented from wobbling pivoted at a site of contact with the fixed lens frame 12 (in other words, a site opposite to the first cutout portion 45 through the central axis O1 of the moving lens frame 21).

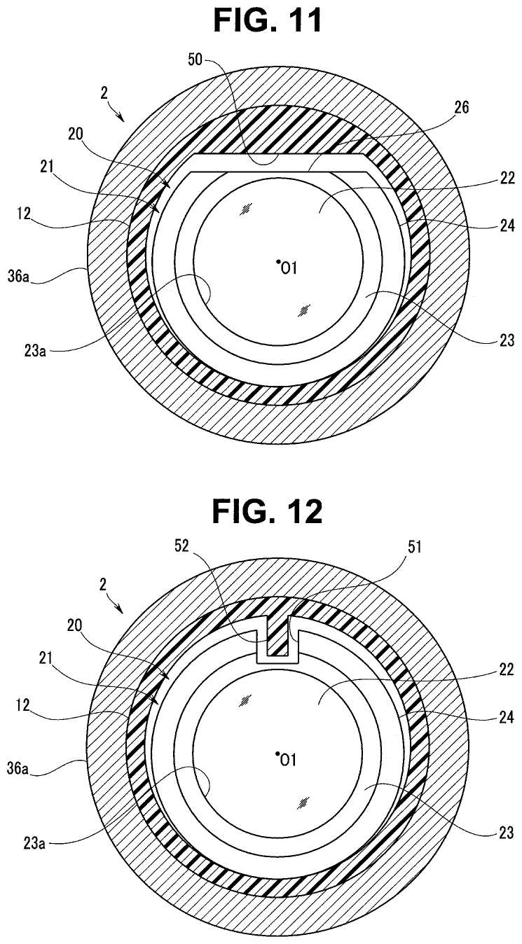

For example, as illustrated in FIG. 11, a rotation restriction portion 50 may be provided on the inner periphery of the fixed lens frame 12 at a position facing the cutout portion 26 of the moving lens frame 21.

With this configuration, a rotational position of the moving lens frame 21 relative to the fixed lens frame 12 can be kept constant.

For example, as illustrated in FIG. 12, as for the shape of a cutout portion, a groove-shaped cutout portion 51 extending in the direction of the optical axis O (central axis O1) may be provided in place of the cutout portion 26 having a planar shape.

In this case, a key-shaped rotation restriction portion 52 may be provided on the inner periphery of the fixed lens frame 12 at a position facing the cutout portion 51 of the moving lens frame 21.

With this configuration, the rotational position of the moving lens frame 21 relative to the fixed lens frame 12 can be kept constant.

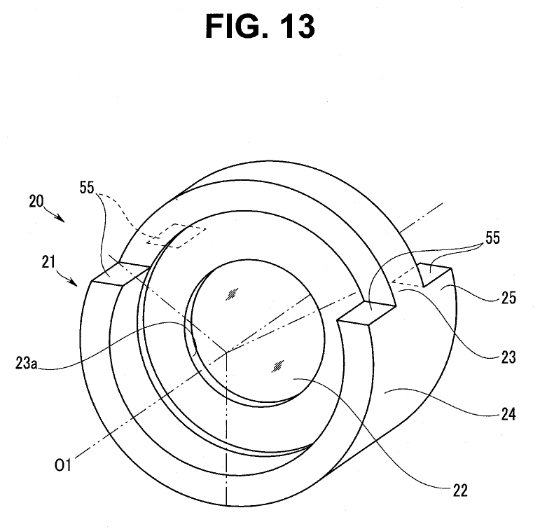

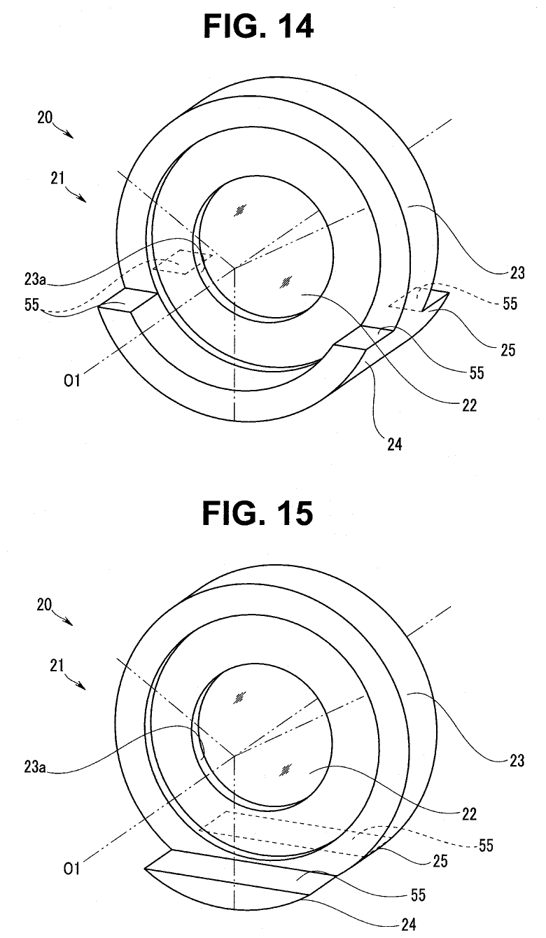

For example, as illustrated in FIGS. 13 to 15, the lens holding portion 23 may be formed to have an outer diameter equal to the outer diameters of the first and second sliding portions 24 and 25.

With this configuration, an outer peripheral surface of the lens holding portion 23 can act as a sliding surface relative to the inner periphery of the fixed lens frame 12. Thus, when a cutout portion 55 obtained by cutting out an optional amount is formed at the first and second sliding portions 24 and 25, as well, a sliding surface of the moving lens frame 21 can contact the inner peripheral surface of the fixed lens frame 12 at at least three points equally spaced from each other by 120° about the central axis O1.

Note that the present invention is not limited to the above-described embodiment but may be provided with modifications and changes in various kinds of manners, and these modifications and changes are also included in the technical scope of the present invention. For example, components of the above-described embodiment and modifications may naturally be combined as appropriate.

What is claimed is:

1. An optical device comprising:
an optical system including a moving lens that is movable in a direction of an optical axis;
a moving frame formed of a magnetic body material and holding the moving lens;
a holding frame formed of a non-magnetic body material in a tubular shape and holding the moving frame on an inner peripheral surface so that the moving frame is movable along the optical axis;
a first magnet and a second magnet disposed separately from each other in the direction of the optical axis on an outer periphery of the holding frame and each having an annular shape; and
a coil wound between the first magnet and the second magnet on the outer periphery of the holding frame,
wherein the moving frame has a non-rotationally symmetric shape about the optical axis.

2. The optical device according to claim 1, wherein the moving frame has such a non-rotationally symmetric shape about the optical axis that magnetic force received from the first and second magnets and the coil at a site in one direction is relatively smaller than the magnetic force received from the first and second magnets and the coil at another site.

3. The optical device according to claim 1, wherein the moving frame includes
a lens holding portion in an annular shape having an inner periphery formed as a lens holding hole,
a sliding portion in an annular shape having a diameter larger than a diameter of the lens holding portion, the sliding portion being provided continuously with the lens holding portion and slidable relative to the inner peripheral surface of the holding frame, and
a cutout portion formed by cutting out part of at least one of the lens holding portion and the sliding portion in a direction orthogonal to a central axis of the lens holding hole.

4. The optical device according to claim 3, wherein the sliding portion includes
a first sliding portion that is slidable relative to the inner peripheral surface of the holding frame on a distal end side of the lens holding portion, and
a second sliding portion that is slidable relative to the inner peripheral surface of the holding frame on a proximal end side of the lens holding portion.

5. The optical device according to claim 3, wherein the cutout portion is formed on a circumference of the sliding portion in a range having a central angle of 120° or smaller.

6. The optical device according to claim 3, wherein the cutout portion has a planar shape.

7. The optical device according to claim 3, wherein the cutout portion has a groove shape extending in the direction of the optical axis.

8. The optical device according to claim 3, wherein the holding frame includes, on an inner periphery, a rotation restriction portion facing the cutout portion.

9. The optical device according to claim 3, wherein a central axis of the moving lens held by the lens holding hole is decentered relative to a central axis of an outer periphery of the moving frame.

10. The optical device according to claim 1, further comprising a yoke provided on an outer periphery of the coil on the outer periphery of the holding frame and configured to amplify magnetic force generated at the first and second magnets and the coil.

11. An endoscope comprising an optical device, the optical device including: an optical system including a moving lens that is movable in a direction of an optical axis; a moving frame formed of a magnetic body material and holding the moving lens; a holding frame formed of a non-magnetic body material in a tubular shape and holding the moving frame on an inner peripheral surface so that the moving frame is movable along the optical axis; a first magnet and a second magnet disposed separately from each other in the direction of the optical axis on an outer periphery of the holding frame and each having an annular shape; and a coil wound between the first magnet and the second magnet on the outer periphery of the holding frame, wherein the moving frame has a non-rotationally symmetric shape about the optical axis.
